(19) United States
(12) Patent Application Publication
(10) Pub. No.: US 2018/0070864 A1
(43) Pub. Date: Mar. 15, 2018

(54) METHODS AND DEVICES FOR ASSESSING A CAPTURED MOTION

(71) Applicant: Matthew Schuster, Portland, OR (US)

(72) Inventor: Matthew Schuster, Portland, OR (US)

(21) Appl. No.: 15/611,774

(22) Filed: Jun. 1, 2017

Related U.S. Application Data
(60) Provisional application No. 62/354,036, filed on Jun. 23, 2016.
(60) Provisional application No. 62/344,854, filed on Jun. 2, 2016.

(51) Int. Cl. A61B 5/11; A61B 5/00

(52) U.S. Cl. CPC A61B 5/1128; A61B 5/0077; A61B 2576/00; A61B 5/7275; A61B 5/7221; A61B 5/7264

(57) ABSTRACT

An analyzed motion of a moving system is assessed to estimate an aspect of the moving system. The aspect is estimated using a relationship formed between the aspect and motion data of the moving system. The relationship may be formed with a predictive algorithm which analyzes a plurality of motions of the moving system to form the relationship.

### BACKGROUND OF THE INVENTION

The present invention relates to the motion capture methods and devices and methods and devices for assessing a captured motion. Body motion tracking and body pose estimation have historically been accomplished using 3 main techniques: Optical systems using markers on the body, optical systems not requiring markers, and non-optical inertial based systems.

Optical systems requiring markers are traditionally very cumbersome to use requiring several carefully positioned and calibrated cameras to capture the motion of special markers attached to the subject. Two or more cameras are used to triangulate the 3D position of these markers, which is then translated into 3D motion, and pose information.

Marker-less optical systems have been developed with recent advances in computing speed and machine learning technology enabling the emergence of marker-less optical systems which are able to take raw optical information from multiple cameras positioned around the subject, recognize the human form in each frame using machine vision techniques and integrate this information into a 3D model of the body pose and motion. Current marker-less optical systems suffer from two significant drawbacks. First, they still require the use of multiple cameras making implementation cumbersome in uncontrolled environments. Second, the measurement precision is generally still too low to be of use in Biomechanics research or clinical assessment and treatment.

Inertial based systems use sensors attached to the body which measure 6 Degree of Freedom rotational rates at numerous positions on the body (i.e. ankle, thigh, wrist, head, etc.). This rotation information is then transferred (via wires or wirelessly) to a computer for processing and aggregation.

Inertial systems are less cumbersome than optical systems because they do not require multiple cameras or special markers attached to the subject. Further, they are typically much more accurate in motion tracking. Inertial systems can currently only measure relative body motion and position. That is, they cannot measure the absolute position of the body relative to the ground plane, nor can they give any information about absolute direction of motion. As a result, these positional errors tend to compound over time resulting anomalies. Further, the accuracy of the aggregate rotational information is directly related to the number of sensors attached to the subject.

### SUMMARY OF THE INVENTION

The present invention is directed to methods and devices for motion capture and for assessing a motion of a captured moving system and methods and systems for measuring and tracking a user's movement (when the moving system is a person) using a set of sensors.

A relationship is defined between an aspect of the moving system and the motion data of a first sensor location. The first sensor location and the aspect may be generated from real motions with inertia measurement units. In this sense one of the sensors becomes a “slave” to another sensor in that one sensor is able to estimate an aspect of the other sensor. The relationship and estimation are defined in quaternion form.

The relationship may be defined using a predictive algorithm and a plurality of motions in quaternion form. For example, the motion data may be analyzed with a neural network to form the relationship. Other predictive algorithms include PGM, support vector machines, random forest and K nearest neighbors (KNN). Maintaining quaternion form may be beneficial as explained herein. The motion data may also be processed to a reduced motion data size in quaternion form.

More than one estimation may be performed on the same person (moving system). For example, 1-4 sensors may be intentionally omitted as described below. In a specific example, relationships may be formed between the motion data and aspects related to the upper arms and upper legs of the person. As will be discussed further below, the motion data for these locations may be estimated with the relationship formed between the other sensors.

The relationship may be defined with respect to any number of sensors. For example, even a single sensor may be used if a relationship is formed between, for example, an upper leg and a lower leg. Defining the movement of one of the upper or lower leg sensors relative to the other may be possible for a squat. Although a squat is a simple movement, in actual fact, the knee joint changes in orientation in a dynamic manner which may permit a relationship with just one sensor. In another example, two or three sensors may be used to define a relationship with an aspect of a moving system with, for example, the upper arm. A lower arm sensor, a shoulder sensor and optionally a torso sensor may be used to define the relationship with the upper arm orientation.

Although the present invention may be used for defining relationships with adjacent structure (joints) on the moving system, the present invention also provides advantages in that distant relationships may also be used to define the relationship. For example, a golfer may be interested in the relationship between the golfer's hands and arms as they relate to the lower leg(s). The present invention provides the ability to form such relationships. Thus, the ability to form relationships with discontinuous/distant structures of the moving system is possible.

In use, the relationship may be used to estimate motion of the moving system for lost sensor data or omitted sensors. The estimated aspect may also be compared to measured aspects derived from measurement corresponding to the estimated aspect to error check the results and current condition. Relationships may be formed for all sensors to check for errors, such as compounded errors in position, for all other sensors. As used herein, the measured aspect may, of course, include the measured sensor data together with a prior position, velocity, or acceleration to obtain a new position, velocity or acceleration. The estimated aspect may also be compared to the measured aspect when the relationship has been formed with a modified version of the moving system.

The relationship may be defined using captured real motion data. For example, the moving system may be a person who performs a plurality of motions which are captured (recorded) and the relationship is formed using this motion data. The motion data may be used to define the relationship using a predictive algorithm such as those described herein. The relationship may be formed during a learning phase prior to the motion data capture event. Once the relationship is defined, the captured (or “analyzed”) motion is recorded with the captured moving system having a first sensor positioned dynamically in at least approximately the same location as the first sensor location.

The aspect of the motion data may be derived from a measurement of one or more of the sensors such as orientation data in quaternion form. For example, the aspect may be an angular displacement (or cumulative sum thereof) derived from integration of the acceleration data. As used herein, the term “derived from” may mean the measured value itself or any mathematical manipulation of that data to compute other values such as total displacements to determine an orientation. The aspect may also be any other value for example the aspect may be derived using an angular displacement, speed or acceleration measured or calculated using measurements of the first sensor. Furthermore, the value may be a cumulative value such as a cumulative angular displacement from a predetermined or selected start value. For example, a patient with a knee injury may be monitored for total angular displacement of the knee or to determine whether a proper squat has been accomplished in physical therapy. The minimum angle and maximum angle also being of interest for monitoring the knee as explained further below in connection with another optional feature of the present invention.

The motion data may also be created or derived without any real motion capture associated with or used to form the relationship. Alternatively, the relationship may be formed using motion data of the moving system itself. The user may be connected to a first set of sensors that measure the user's first movement(s). The input from the sensors is analyzed and relationships are developed between the movements and the sensor inputs. The user may then perform a second or “analyzed” movement using a second set of sensors that may have the same or fewer sensors than the first set. In some embodiments, this may be the removal of some of the first set of sensors to create the second set of sensors. The inputs from the user's second movement are analyzed and additional information regarding the movement is calculated and estimated using the relationship(s) defined from the first movement. The information may be added to display the user's second movement or track, store, or compare the movement. Alternatively, the second movement may be further analyzed to determine differences between the first movement. Comparison may be for athletic performance or rehabilitation.

The estimated aspect may be used to supply missing data due to data loss (drop out or other problem with data). The present invention also provides the ability to use fewer sensors with the sensor location for the aspect having the defined relationship being intentionally omitted. When encountering the problem of missing data during the analyzed motion, a potential advantage of the present invention is that the relationship may be established in advance and estimation of the missing data may be undertaken during the analyzed motion. For example, the motion data may be displayed during the analyzed motion so as to have “real time” application. The relationship may also be used to check for errors during the analyzed motion as well by comparing the estimated value with a measured value derived from the sensor(s). The system estimates the data and optionally displays and may even transmit the data to a remote location all while the analyzed motion takes place. The analyzed motion may be as short as 3 seconds or even 1 second for local display during the analyzed motion (and even transmission over the internet).

To create relationships between the sensor inputs and the user's movements a number of techniques may be used. For example, in some embodiments a probabilistic graphical model (PGM) may be used to establish relationships between inputs and movements. Alternatively, in other embodiments a K Nearest Neighbors (KNN) technique or the like may be used to establish relationships between inputs and movements. Any number of other suitable techniques may be used. In some embodiments, a machine learning technique such as Probabilistic Graphical Models (PGM), Neural Networks, Support Vector Machines, non-linear regression, or K-Nearest Neighbors is used to model the relationships between inertial data captured by sensors worn on the human body. The model is then used to predict the state of a missing sensor (or sensors), fill in missing data during sensor (or sensors) failure or drop out, and to error check the output of a sensor (or sensors) to insure reliable and proper operation. Additionally, any suitable other method may be used.

In some embodiments, what is invented is a method and system for capturing, transferring, and modifying/transforming data sets among a network of devices to create a partial or systemic relativistic model(s) of motion. Such a method and system may include at least a single capture device which captures raw data sets with sensors and data acquisition units, or equivalent, and transfers such data sets to a processing unit on the capture device or on a separate master device via wired or wireless data transfer. Upon data set capture by the processing/capture unit, the data set may be modified and/or transformed to a modified data set. Such modifications may include transforming data set coordinates (i.e. relative Cartesian, polar, etc.), units (i.e. metric, English, etc.), direct modification of parts of the motions themselves, missing or additional motion data augmentation based on probabilistic or artificial intelligence computational models or equivalent. Such capture and transformation of motions may be used to build a general motion library. This library may be comprised of specific modified or unmodified past motions as well as unique computational, probabilistic kinematic models that can be used for predicting the positions of un-sensored kinematic elements on the body being measured. These types of files will be considered motion master profiles as compared to data that is currently being captured, modified, compared or streamed. These files may be used to form relationships in quaternion form between the aspect and up to all of the other sensors.

As a general example, an inertial measurement unit (IMU) (slave) device is worn on an appendage or part of the object (moving system), which captures accelerations (e.g. accelerometer), and angular velocities (e.g. gyroscope), and transfers such captured data to a master device which processes and transforms the data to useful or augmented data sets that create master motion files. Some of the data processing may occur on the IMU prior to transferring to the master device. Such an unmodified or modified data set may then be used to directly compare the motion standards of various other such motion events, whether or not previously modified.

In another more specific example at least one slave device is worn at various body members of patient undergoing physical therapy that record accelerations and angular velocities during a leg exercise. Data is captured, processed, buffered by each slave device and dynamically transferred to a master device, or at a later time. Parameters or aspects such as accelerations at various points of the leg exercise, time of back-movement/front-movement, leg twist, etc., may then all be determined by data processing and transformation to a coordinate system relative to the patient's body. Such parameters or aspects may then be easily compared among various leg exercises by the patient or among various exercises by various patients. The present invention allows the user to capture a previous motion file, modify it automatically or manually, and then compare other motions to that profile. Further, no system or method currently known will currently report significant, accurate information on un-sensored body members (whether by IMU or camera fiducial) that are more than one kinematic link away from a sensored kinematic element. For example, today, in order to accurately predict full body motion, typically only adjacent kinematic elements are used to estimate motion. The present invention may form relationships between the lower leg and torso for example.

In some embodiments, what is invented is a method and system for comparing a (slave, or equivalent) modifiable motion standard (analyzed motion) to a modifiable master motion standard which forms relationships in the motion data. Such a method and system comprises a processor having executable code for comparing at least two motion capture event data sets. Such data sets may be single motion capture events, averages of multiple motion capture events, manually or automatically augmented or adjusted motion standards, or artificially constructed motion capture events. Slave and master capture event data sets may be first analyzed and transformed algorithmically to create a motion standard using a computational, probabilistic modeling technique such as a Bayesian network for determining and comparing key comparators. A motion standard comprises spatial and time coordinates to map out and represent the motion capture event(s). Key comparators comprise derived parameters that describe or define their originating data set in parts or in whole. Key historic and/or current comparators may be compared by means of standard statistics or a higher order statistical panel (i.e. second and third order statistics), etc. Additionally, data compression techniques as well as dimensionality reduction techniques for comparing “two signals” may be employed.

Using machine learning to discover differences and unique characteristics of motion standards (one being a set of motions for which the relationship(s) are formed and the other being the analyzed motion(s)) may also be employed for automatic, or objective, etc. determination of differences between motion standards, etc. (i.e. as compared to human or subjective evaluation of differences among motion standards). Differences may be described using a statistical panel (such as higher order statistics), visual methods, etc. For example, differences may be compared as percentage increase/decrease/differences, absolute increase/decrease/differences, multiples, etc.), as visual overlays of the motion standards (static or dynamic), etc. A user interfacing dashboard on website or personal computing device, or equivalent, may present a user with a means of modifying the slave or master motion standard and monitoring key comparators. Such modifications include artificially modifying a numeric key comparator directly (which may result in an output of necessary motion modifications to acquire such modified key comparators), artificially modifying a motion standard by means of a multidimensional drag and drop of various kinematic element or joint locations or coordinates along the motion standard (which may result in derived key comparators based on the updated motion), with the software algorithmically maintaining continuity in the profile.

In some embodiments, what is invented is a method and system for creating a virtual avatar of a fully or partially moving motion (i.e. relative to “grounded” i.e. earth grounded) target object (i.e. human body) which receives and mimics motions captured via a motion capture system. Such an avatar may reflect real time motion capture, or be used to replay motion that has previously occurred, or be used to demonstrate a desired motion as a standalone or compared to a current or previous motion. Such a system would allow for full representation of the moving object through the avatar, with motion representation derived from any combination of the following: actual motion as captured by at least a single motion capture device on the motion target (i.e. inertial measurement unit (IMU), or equivalent), algorithmically derived (estimated) motions based on actual motions captured by the motion capture device(s) working in sync with a motion library (both of which may be modifiable by the user), and/or algorithmically derived motions based on input from any combination of a camera, IMU(s) and probabilistic computational models. The avatar may be fully mobile or certain parts or kinematic elements of the motion target may be represented as static if insufficient data is available. The backdrop of the camera may be used as the backdrop of the virtual avatar to create a more complete virtual representation of the target object and its motion through its environment. Such represented motion would allow for creating an empirically derived complete motion standard of a motion target, and ideally represent “life-like” motion for the case of human motion targets, or equivalent. Such virtualization also allows for motion captures to be streamed or sent to remote locations, for real time analysis and recommendations for improvement/change/etc. of such motions (ref. comparators mentioned herein).

In some embodiments, what is invented is a method and system by which sensors (IMU(s) and Camera(s)) and computational and or probabilistic models are used to create a motion standard which can be stored to become a modifiable master motion standard with associated key performance statistics. This motion standard can be directly displayed as an avatar on a screen or VR device in real time or in playback mode. The system may allow display of the avatar over or in a split screen mode next to another motion standard. For example, in the case of a workout video, the leader (either pre-recorded or live) would be shown on one half of the screen and the actively monitored person's avatar displayed adjacent to them. This may enable a real-time virtual remote yoga class, or other such group event, with participants seeing and following a teacher with their comparison data being dynamically displayed as their real-time avatar is being displayed simultaneously. Real time statistics of key motion elements or statistics could be dynamically displayed and feedback given based on these. In another instance, a physical therapy patient may be performing remote rehabilitation and a split video representation may be useful to a clinician and the patient as they instruct on, or review live or pre-recorded rehabilitation exercise motions and compare key progress and or performance data. The historical metric tracking dashboard discussed above might be similarly useful in this scenario as well.

In some embodiments, what is invented is a method and system by which sensors are used to estimate the position of the pivot point of a dynamic joint. Pivot points of some physical joints, such as anatomical joints within humans like the knee or shoulder joint, are not stationary and move dynamically as the joint is rotated. Sensors can measure the location of the pivot point of the joint dynamically as it moves with the movement of the joint.

In some embodiments, what is invented is a method and system for tracking objects which can be used in virtual reality environments. Multiple sensors on an object, such as a human, can be used to monitor and track the position, orientation, and anatomical movements of a user and mapped into a virtual reality.

There are many situations where measuring and displaying a user's motion is useful. For example, in the case of physical therapy it is important for a trainer to be able to monitor and track specific movements of a patient. In some instances, the patient may be in a different location and the trainer may be trying to monitor the patient remotely. In other instances, the trainer may want to track a patient's movements over time. In both cases having accurate representation of the patient's movements is important. Simultaneously, reducing the number of sensors required to measure or display a patient's movements may be beneficial since this may reduce the required software or hardware resources.

The present invention is similar to a pure inertial system in that sensors (gyroscopic rotation sensors and/or multi-axis accelerometers, etc.) are attached to the subject. In one feature of the invention a Bayesian probabilistic framework is then used to capture, learn and describe the probabilistic relationships between the motion (i.e. rotation, acceleration, etc.) information at different points on the body. There are several advantages to this approach. First, it may reduce the number of sensors required to be worn by the subject, second it will be very tolerant of bad data or data drop-outs by one or more sensors, and third, other sources of body tracking information (single perspective camera for example) can easily be integrated into the probabilistic model to increase overall system accuracy.

Example use case: Motion and Pose analytic tool. In the context of physical therapy, minimizing the number of sensors may be important. It is critically important that the patient perform the exercises correctly and that the physical therapist is able to review the exercises for correct execution and for changes in range of motion, with minimum impact to patient's rehabilitation routine. Using a motion capture system, the exercises can be performed in a supervised setting and recorded as a baseline or prototype for the patient. This can be used to create a motion standard. When the patient next performs the exercise while wearing the motion capture apparatus, the new motion can be compared with the prototype or baseline and learning model generated motion standard with a relationship between some aspect of motion and the other motion data to look for deviations. When looking for such deviations, a clustering algorithm such as K-means, or Hierarchical clustering can be applied to the motion capture time series data. The data can be divided into an arbitrarily large number of clusters. This clustering represents a dimensionally reduced view of the movement in question, where the number of clusters chosen represents the fidelity of the view. This reduced dimensionality view can then be used to compare against the prototype view, and allows the practitioner to pick out key aspects of the motion and efficiently look for those in the clustering profile. These parameters can be added to a motion standard.

The first sensor may be an inertial measurement unit (IMU) including a first inertial sensor, a second inertial sensor, and a third inertial sensor and a global directional sensor. The global directional sensor may be at least one of a gravity sensor and a magnetic sensor or both. The first, second and third inertial sensors may measure rotational accelerations in orthogonal orientations as is known. Furthermore, the IMU may integrate acceleration values to achieve velocities and displacements as is also known. As will be described further below, the IMU may transmit these values to a processor which performs the estimations and other assessments described herein. The IMU may also include a first accelerometer, a second accelerometer, and a third accelerometer to measure accelerations in orthogonal directions. Thus, the IMU may have three accelerometers, three gyroscopes and optionally magnetometers and/or a gravity sensor. The accelerometers and gyroscopes are also placed in a similar orthogonal pattern measuring rotational position in reference to a chosen coordinate system.

In practice, the person may perform motions immediately in advance of performing the analyzed motion or at some time prior in a known condition (healthy, post-op, transient state during physical therapy). The motion data may also include data from prior captured motions such as those that were analyzed motions on a prior day. In this manner (as will be described below) the relationship can be refined over time if the motion system is in a transient state. For example, a knee ligament replacement may change in dynamic character in the first few months during which the motion library may be altered using or adding the analyzed motions to the motion library which defines the plurality of motions.

The motion system may also be not the same as the captured motion system. For example, the motion system may be a famous professional athlete or performer or may be a modified or hybrid version of the capture moving system or may be an artificial/animated object. The relationship between an aspect of these systems and the motion data of the moving system may be defined in advance without requiring a determination using motion data.

The relationship may also be an algorithmic relationship, a tabular relationship, or may be defined with the moving system being a modified version of the moving system. For example, the plurality of motions may simply be a modified version of a real motion of the moving system (such as a person). The relationship may also be determined with the modified version of the moving system being modified toward a target motion.

The motion data is substantially retained and manipulated in quaternion form. The relationship between the aspect of the motion data and other parts (or all) of the motion data is estimated in quaternion form with each coefficient being estimated. The relationship may be determined with the relationship between the aspect and all of the motion sensors in the motion data less no more than one or two sensors. Stated still another way, the relationship may be between the aspect and at least 75%, or all, of the motion sensors in the motion data. In a specific example, the relationship may be determined with the aspect related to motion for a lower arm and a lower leg. The person may wear either an upper leg sensor or a lower leg sensor with the relationship being defined accordingly. For example, the aspect may be related to a value derived from a motion measurement of a torso sensor attached to a torso of the moving system which may be used to estimate values for a common torso sensor (whether present or intentionally or unintentionally missing data).

The present invention may be carried out with a first processor carried (supported) by the moving system which may perform estimations of the aspect and error check and correction prior to transmitting the motion data and estimated motion data to a main processor (which may be independent of the moving system). The moving system may include several processors with each coupled (preferably by hard wire) to one or more sensors for each estimating an aspect based upon a reduced or regional set of sensors. An advantage of providing one or more processors on the person (moving system) which estimates the aspect computing demand on the main processor is reduced which may improve performance of a high population virtual environment. The distributed processing at each of the moving system unburdens the main processor and provides advantages for environments with multiple users in the same environment.

Further advantages will become apparent from consideration of the ensuing description and drawings.

### BRIEF DESCRIPTION OF THE DRAWINGS

FIG. 1A shows an outline of a human body with full sensor set

FIG. 1B image shows an outline of a human body with a reduced sensor set

FIG. 2A shows an outline of a human body with full region specific sensor set

FIG. 2B shows an outline of a human body with a reduced region specific sensor set, with circular nodes indicating intended sensor location and “X” nodes indicating locations required to complete a full region specific sensor set

FIG. 3 shows example data/computational flow

FIG. 4 shows example data/computational flow for pose estimation and body tracking

FIG. 5 shows example data/computational flow for pose estimation and body tracking with reduced sensor set input

FIG. 6 shows example data/computational flow for pose estimation and body tracking with region specific sensor subset input

FIG. 7 shows example data/computational flow for pose estimation and body tracking with reduced region specific sensor subset input

FIG. 8 shows example data/computational flow for pose estimation and body tracking with reduced sensor set input

FIG. 9 shows a full sensor set

FIG. 10A shows an outline of a human body with full sensor set

FIG. 10B shows an outline of a human body with a reduced sensor set

FIG. 11A shows an outline of a human body with full sensor set, with circular nodes indicating intended sensor location

FIG. 11B shows an outline of a human body with a reduced sensor set

FIG. 12 illustrates an example information flow in the present invention.

FIG. 13 illustrates an example information flow in the present invention.

FIG. 14 shows a system with a processor carried by the user which estimates motion data for the user with the sensor data and transmits the sensor data and the estimate to a main processor.

FIG. 15 shows a comparison of a first coefficient of estimated in accordance with the present invention compared to a measured value derived from a sensor at the estimated location.

FIG. 16 shows a comparison of a second coefficient.

FIG. 17 shows a comparison of a third coefficient.

FIG. 18 shows a comparison of a fourth coefficient.

### DETAILED DESCRIPTION OF THE PREFERRED EMBODIMENTS

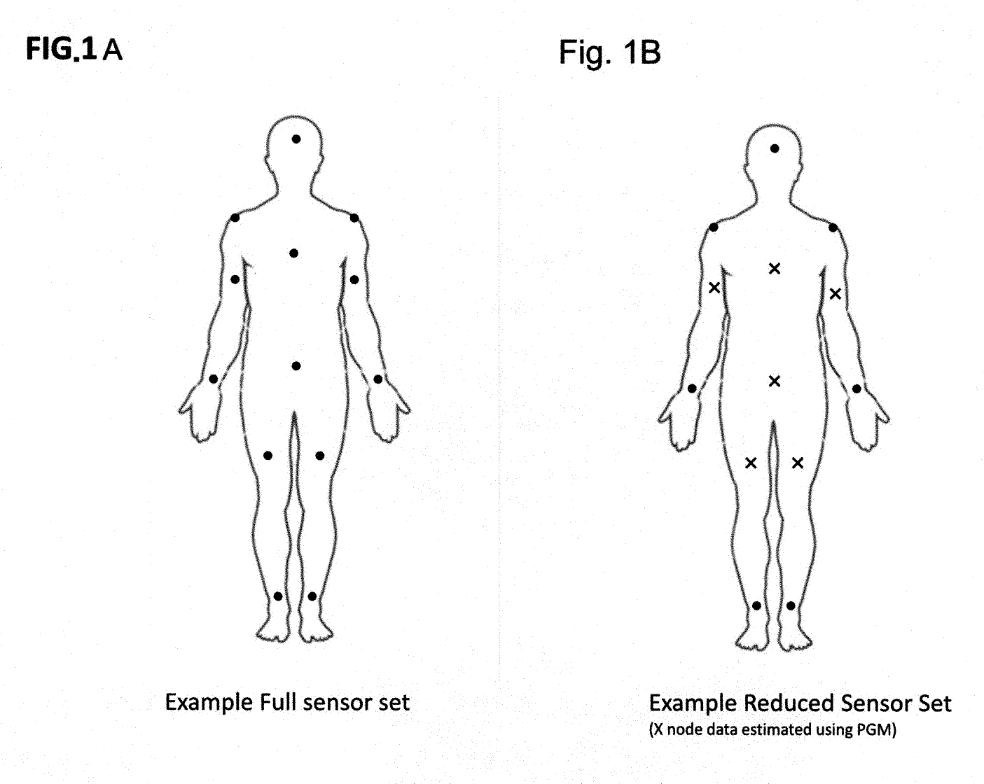

Referring to FIG. 1A, a graphical view of a human with a sensor set. The circular nodes indicate intended sensor location for a full sensor set is defined as the minimum number of sensors required to model body poses and track body motion with acceptable accuracy and resolution given a specific application. We notionally use 13 sensors for clarity in this example.

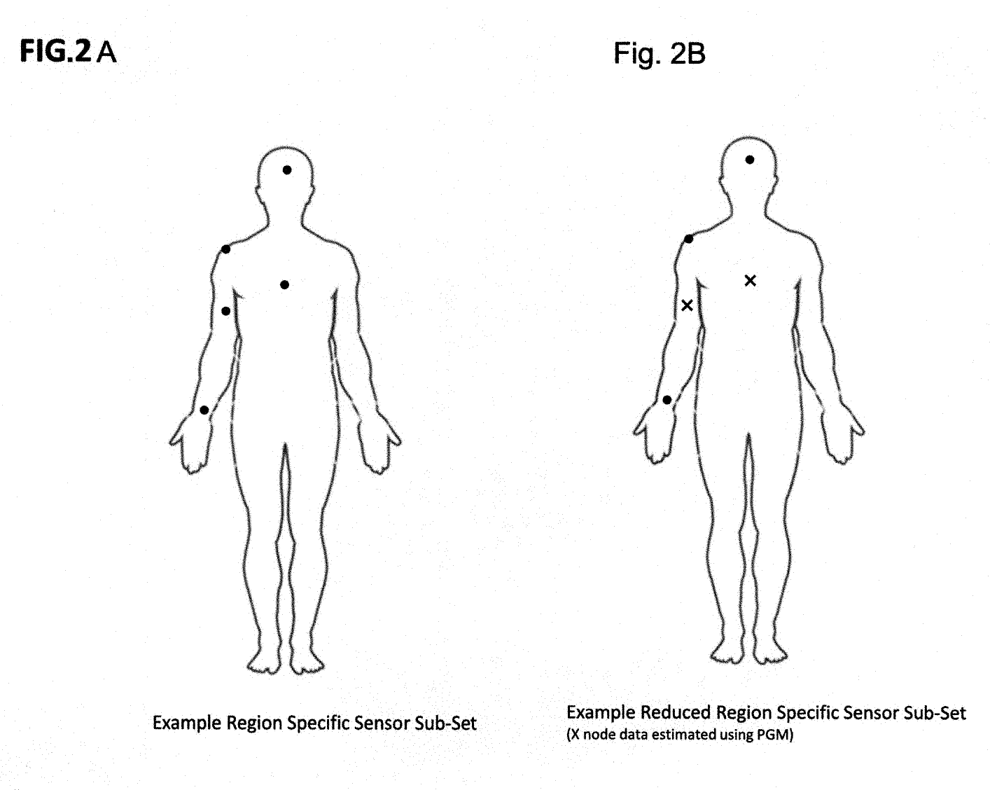

FIG. 1B shows the circular nodes indicating intended sensor location and “X” nodes indicating locations required to complete a full sensor set. FIG. 2A show circular nodes indicating intended sensor location (a full region specific sensor set is defined as the minimum number of sensors required to model a subset of the body which is of primary relevance to a specific motion for poses and tracking body motion with acceptable accuracy and resolution given a specific application. We notionally use 5 sensors for clarity in this example). The missing nodes “X” (FIG. 1B and FIG. 2B) may also include a sensor so that the estimation serves as an error check rather than supplying missing information and such use is incorporated for all embodiments herein.

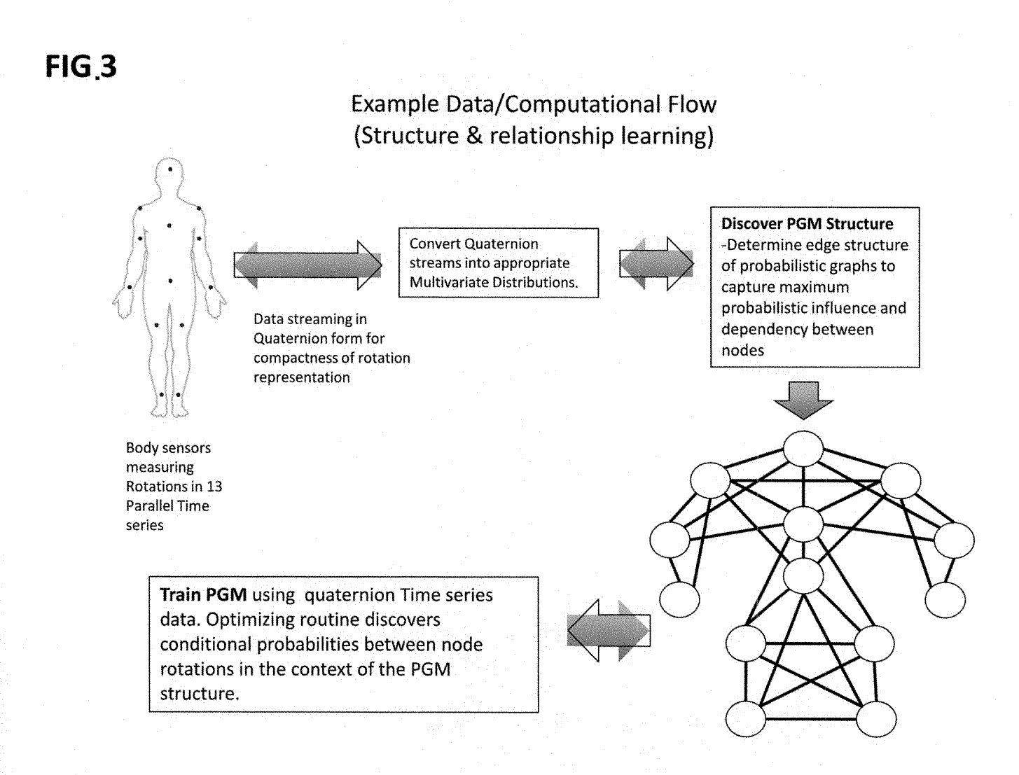

The sensor positions on the body depict an example configuration used to gather simultaneous streams of rotation data. This could be extended to include acceleration data. In this example, the rotation data is streamed in quaternion form. This preserves the coupled nature of the rotation data, avoids degenerate solutions (such as gimbal lock using Euler angles) and is more compact. The Quaternion time series data is then analyzed using a predictive algorithm. For example, the motion data may be converted to multivariate probability distributions for each sensor. This Quaternion distribution data is used to construct a Probabilistic Graphical Model. The graph could be in the form of a Bayesian network (a directed acyclic graph), a Markov Network (an undirected acyclic or cyclic graph), or a variety of other configurations such as a hidden Markov model. The PGM is trained using the Quaternic distribution data. The structure of the graph is informed by the natural dependency relationships of the human body. That is, as one part of the body moves there is a structural dependency relationship to motion is in other parts of the body. The example PGM structure shown in FIG. 3 depicts this sort of relationship. In this example, The PGM shown is an undirected Markov network. The edges between nodes represent the probabilistic influence that motion in one node exerts on the other connected nodes. For example, the rotations measured at the ankle sensors are conditionally related to the rotations of the thigh sensor, which are in turn related to rotations of the torso sensors.

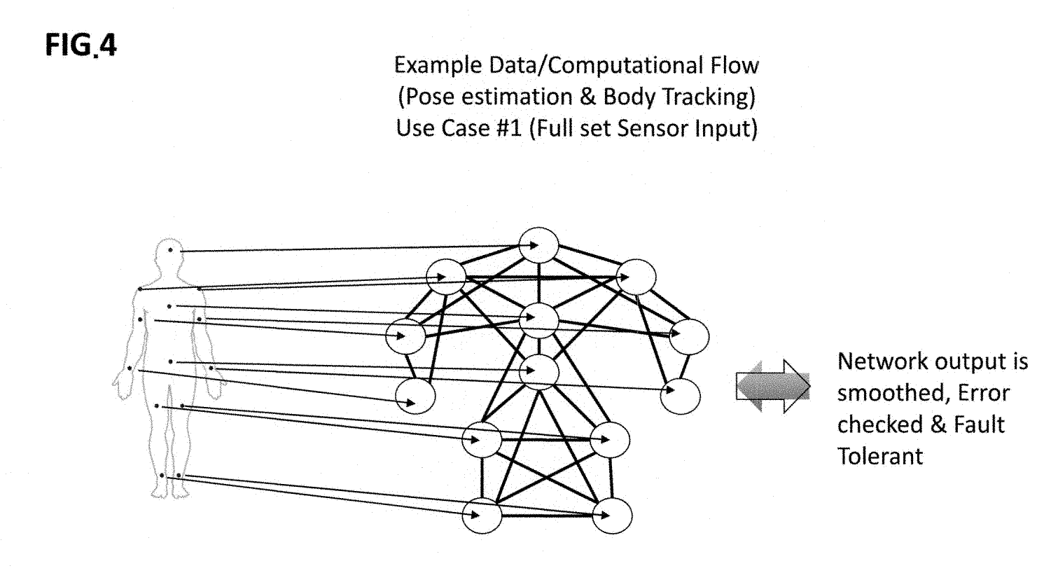

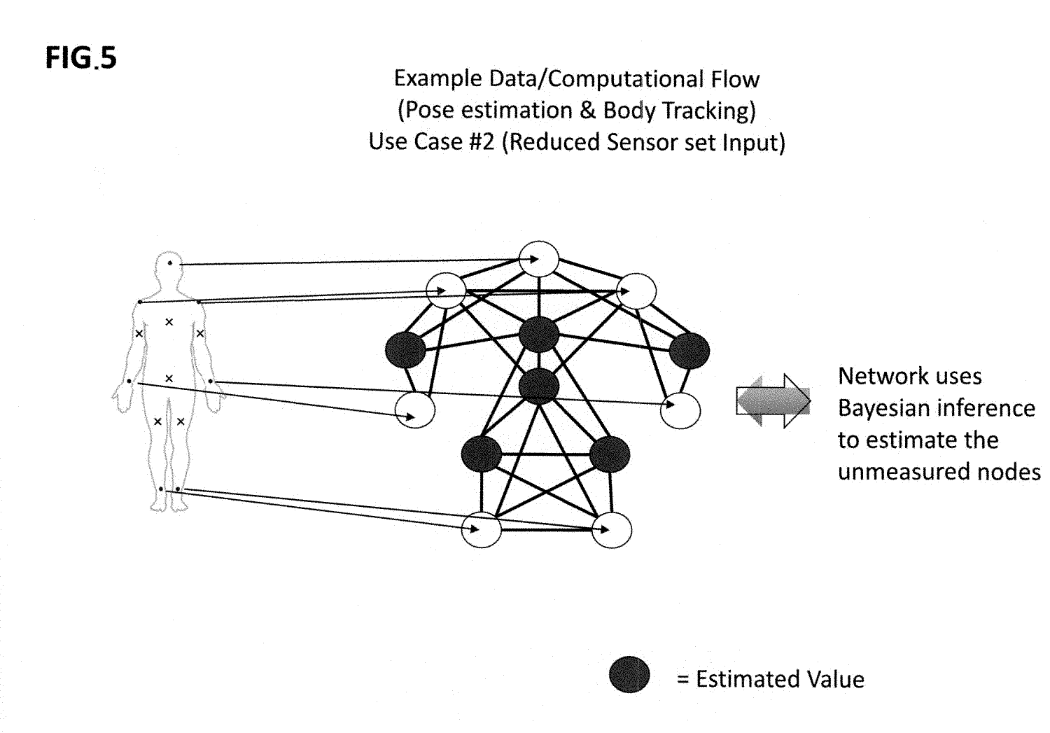

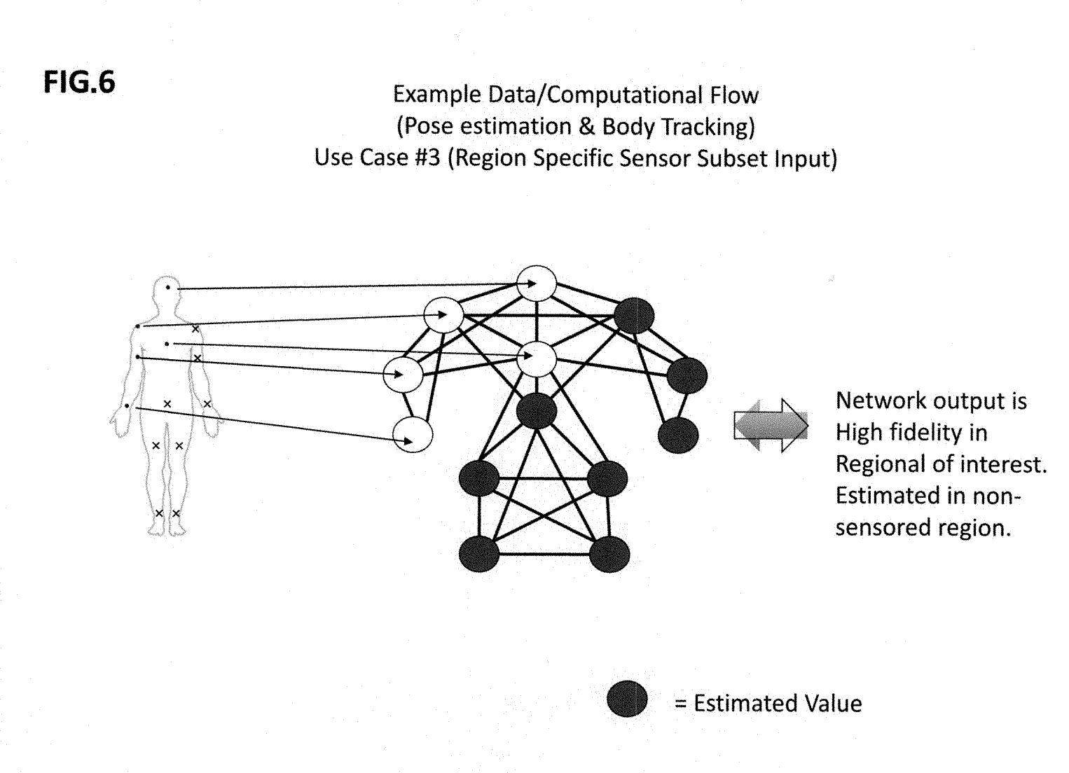

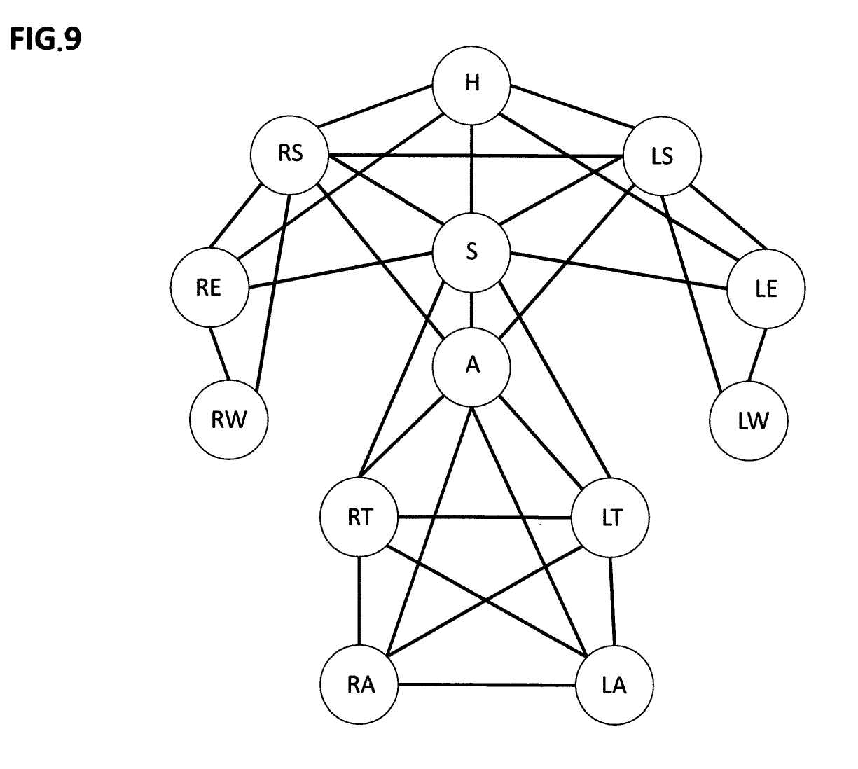

FIG. 3 diagrams the process of using the sensor output to configure and train the PGM. In this example, the subject is fitted with a full sensor set. In this context, a full sensor set is defined as the minimum number of sensors required to model body poses and track body motion with acceptable accuracy and resolution given a specific application. We notionally use 13 sensors for clarity in this example. The 13 sensors are synchronized and produce quaternion time series. The time series data can be viewed as a random variable with a normal distribution. Using a 4×4 real matrix representation, each quaternion data point can be mapped to the space of real matrices. This allows the use of more conventional second order statistics on the quaternion data. Specifically, an optimization algorithm can determine the optimal structure of a Probabilistic Graphical Model capturing maximum probabilistic influence and dependency between nodes (see also full sensor set of FIG. 9). Appropriate training during a training phase and validation and test datasets can then be used to train the network. The probabilistic influence that each node exerts on every other connected node is then contained in the Bayesian conditional relationships in the network. The structured and trained network can then be used to inform the body pose/motion tracking system (FIGS. 4 and 5). An example use case is a Motion and Pose analytic tool (FIG. 4). In the context of physical therapy, minimizing the number of sensors may be important. It is critically important that the patient perform the exercises correctly and that the physical therapist is able to review the exercises for correct execution and for changes in range of motion, with minimum impact to patient's rehabilitation routine. Using a motion capture system, the exercises can be performed in a supervised setting and recorded as a baseline or prototype for the patient. This can be used to create a motion standard. When the patient next performs the exercise while wearing the motion capture apparatus, the new motion can be compared with the prototype or baseline and learning model generated motion standard with a relationship between some aspect of motion and the other motion data to look for deviations.

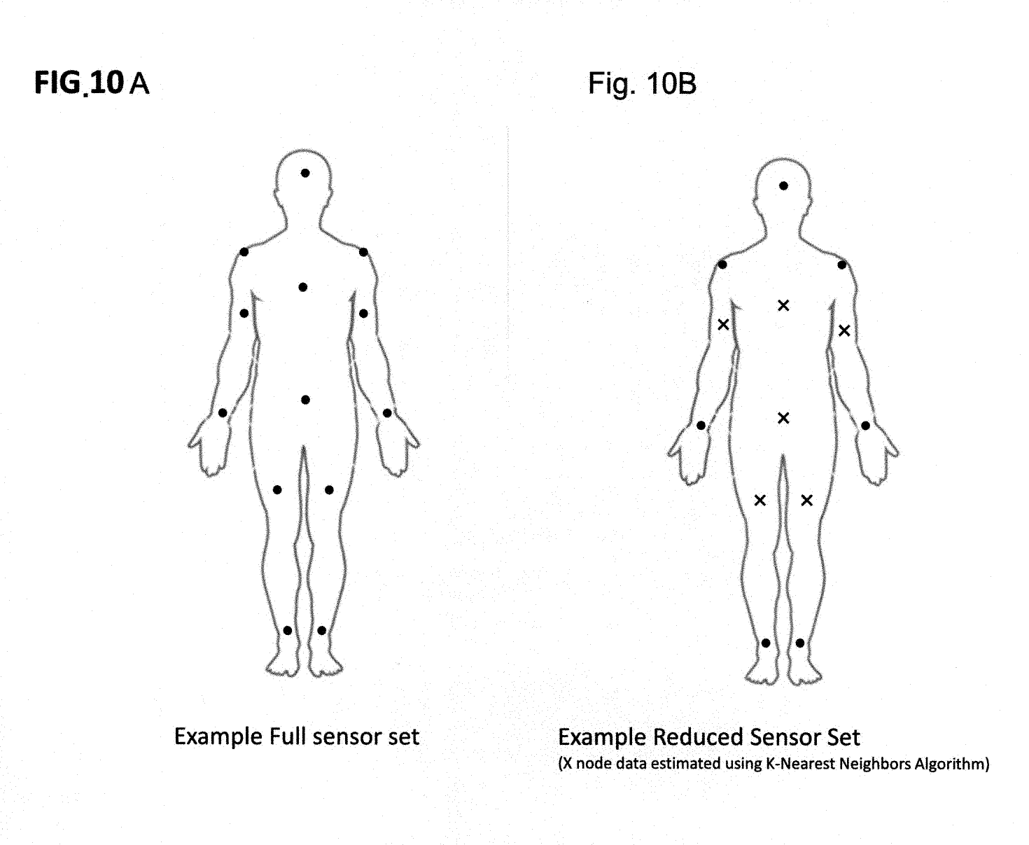

FIG. 10A shows an outline of a human body with full sensor set, with circular nodes indicating intended sensor location (a full sensor set is defined as the minimum number of sensors required to model body poses and track body motion with acceptable accuracy and resolution given a specific application. We notionally use 13 sensors for clarity in this example).

FIG. 10B shows an outline of a human body with a reduced sensor set, with circular nodes indicating intended sensor location and “X” nodes indicating locations required to complete a full sensor set, and of which are estimated using the K-Nearest Neighbors algorithm.

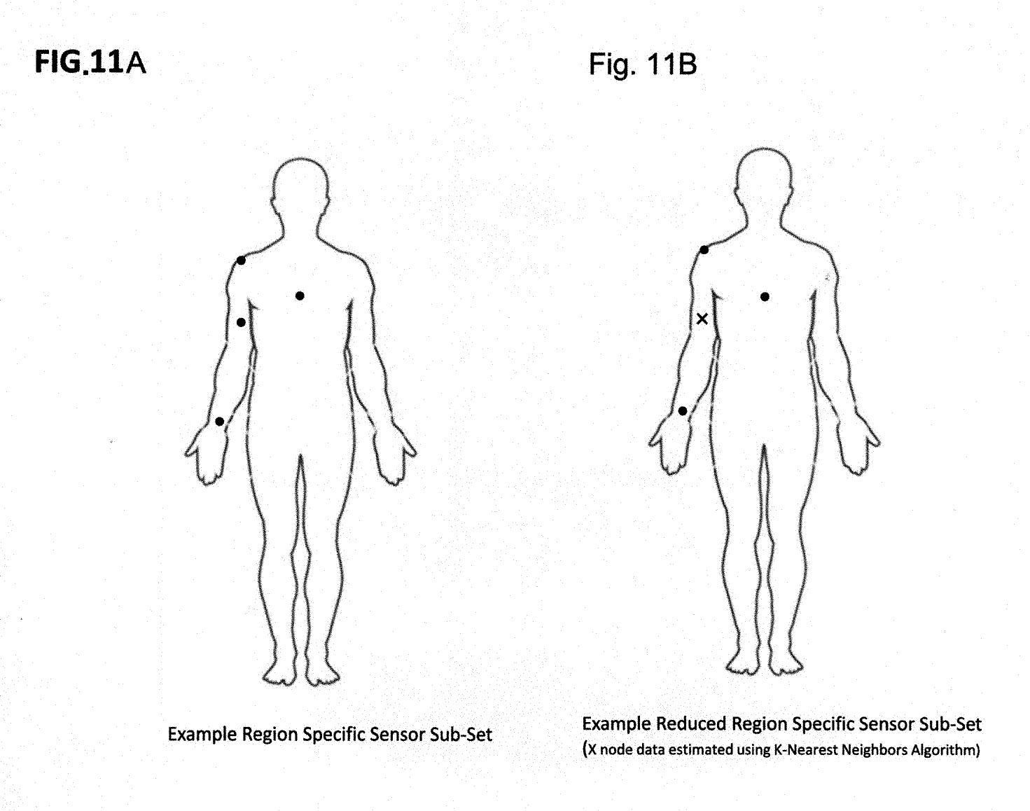

FIG. 11A shows an outline of a human body with full region specific sensor set, with circular nodes indicating intended sensor location (a full region specific sensor set is defined as the minimum number of sensors required to model a subset of the body which is of primary relevance to a specific motion for poses and tracking body motion with acceptable accuracy and resolution given a specific application. We notionally use 4 sensors for clarity in this example).

FIG. 11B shows an outline of a human body with a reduced region specific sensor set, with circular nodes indicating intended sensor location and a “X” node indicating the location required to complete a full region specific sensor set, and of which are estimated using the K-nearest Neighbors algorithm.

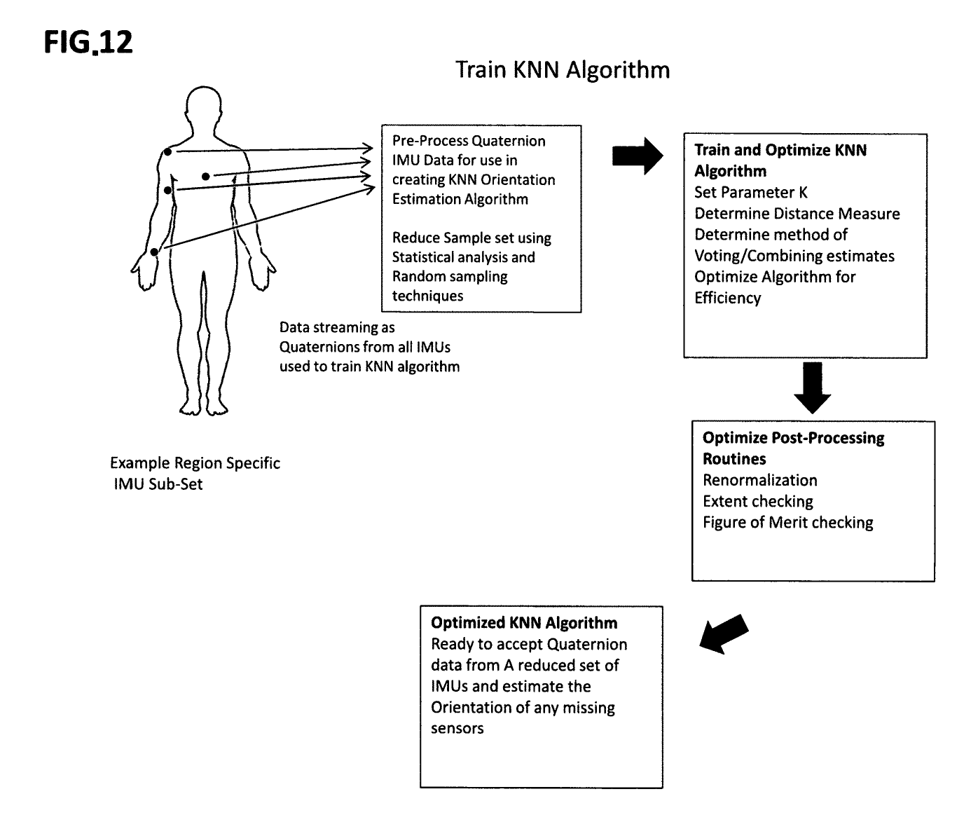

FIG. 12 illustrates an example information flow in the present invention. The sensor positions on the body depict an example configuration used to gather simultaneous streams of rotation data. This could be extended to include acceleration data. In this example, the rotation data is streamed in quaternion form. This preserves the coupled nature of the rotation data, avoids degenerate solutions (such as gimbal lock using Euler angles) and is more compact. The Quaternion data is pre-processed using tools to insure uniformity in the data, look for data errors and drop-outs. The Quaternion data set may also be analyzed and reduced in size using Random sampling techniques such as Simple Random Sampling, Monte Carlo methods, stratified sampling or cluster sampling. The Pre-processed data set is then used to construct a K-Nearest Neighbors orientation estimation algorithm. Human subject specific data is analyzed and used to optimize the parameters and efficiency of the predictive algorithm. Additionally, any required post-processing routines are optimized. These steps result in the creation of a trained KNN algorithm ready to accept Pre-processed quaternion data from a reduced set of IMUs.

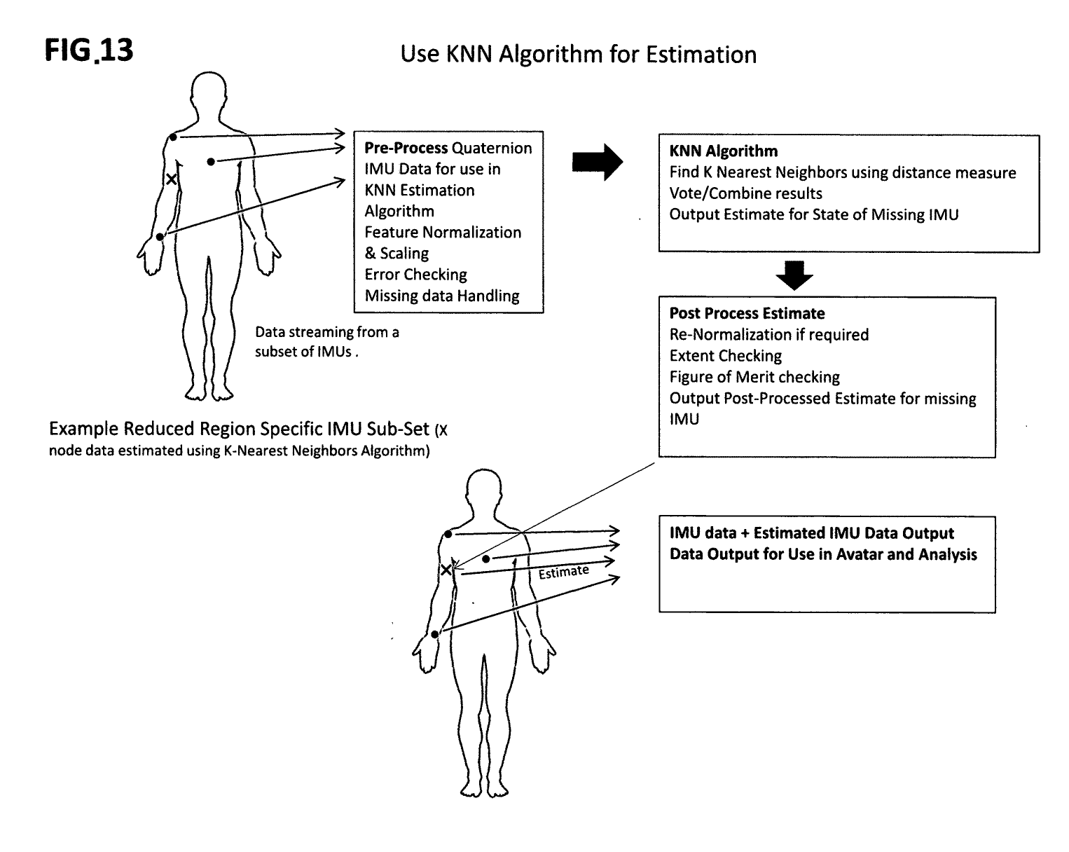

FIG. 13 illustrates an example information flow in the present invention. In this case, a trained KNN algorithm is used to predict the orientation of a missing IMU based on the inputs of a reduced set of IMUs. The IMUs pass raw quaternion data to a Pre-processor which normalizes and scales the data as well as performing error checking and handling missing data. The Pre-processed data is passed to the K-Nearest Neighbors algorithm that then determines an estimate based on the input features proximity to previously learned data using an appropriate distance measure. The estimate is Checked for reasonableness using an extent check, and Re-normalized if required. Additionally, statistics are gathered on the quality of the estimate that are used to further optimize the KNN algorithm, and to report the quality of the estimate. The post-processed estimate is then combined with the IMU data streaming from the present IMU's. The fused measured/estimated data is then output for use in animating an avatar, analysis, storage for later use, or any other appropriate use.

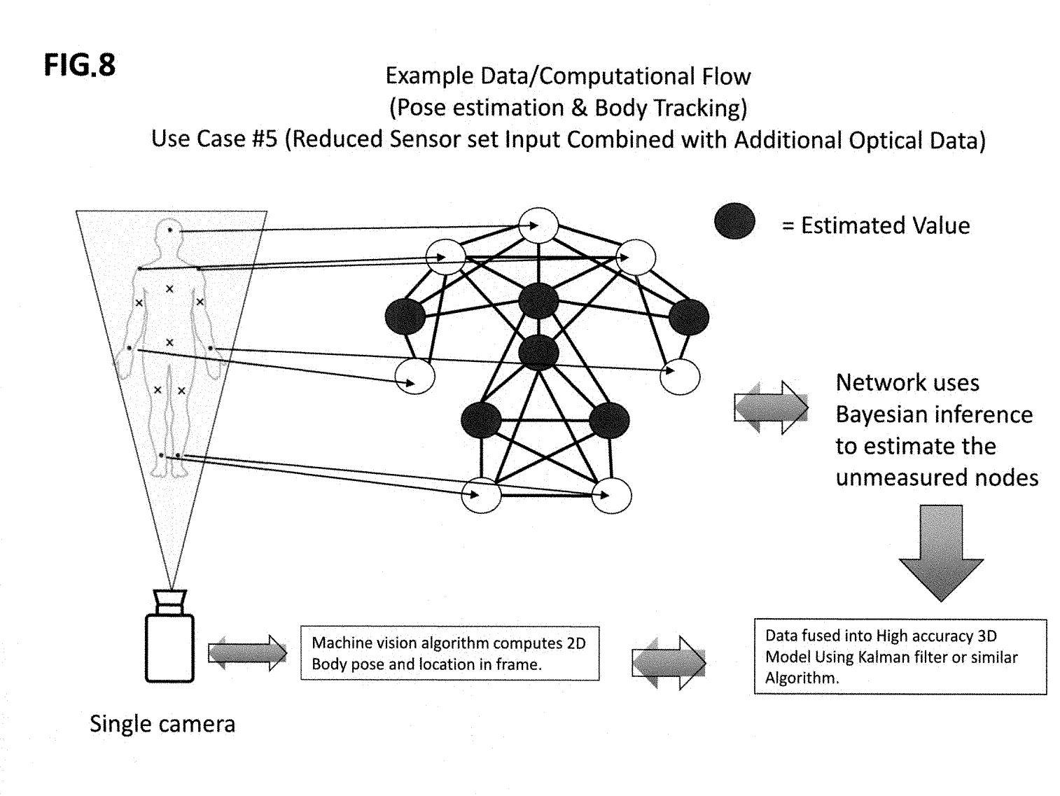

While several embodiments of the present invention are described by example in the above text, the following describes an embodiment where a more complete motion is estimated and created from a partial motion data set with reference to FIG. 8. In this embodiment, a camera is used to recognize that the motion data set is not complete, but any number of other methods may be used and some methods and systems require no camera data.

A system would comprise a motion capture system, a camera, and a computer with graphical interface (inclusive of supporting components for data transfer between motion capture system and computer). An application example of such an embodiment is as follows: A video capture camera records (and may use body, edge, fiducial or other similar tracking technology known to someone skilled in the art of humanoid tracking) a user performing a motion. A user's leg motion is captured via a motion capture devices (IMUs) and transferred to a computer, which may combine the data and then relays the motion of user's legs real time, visually. Software on the computer recognizes motion is not being received for the rest of the user's body (aside from leg motion) by the motion capture system, and references a combination of a motion library, motion algorithm's, and input from the camera to fill in anticipated motion of remainder of user's body and overlay a representative backdrop around the avatar. The end result is a moving avatar that represents a user's complete motion, with high accuracy and recording of all moving parts tracked by the motion capture system, and representative motion filled in by the aforementioned system.

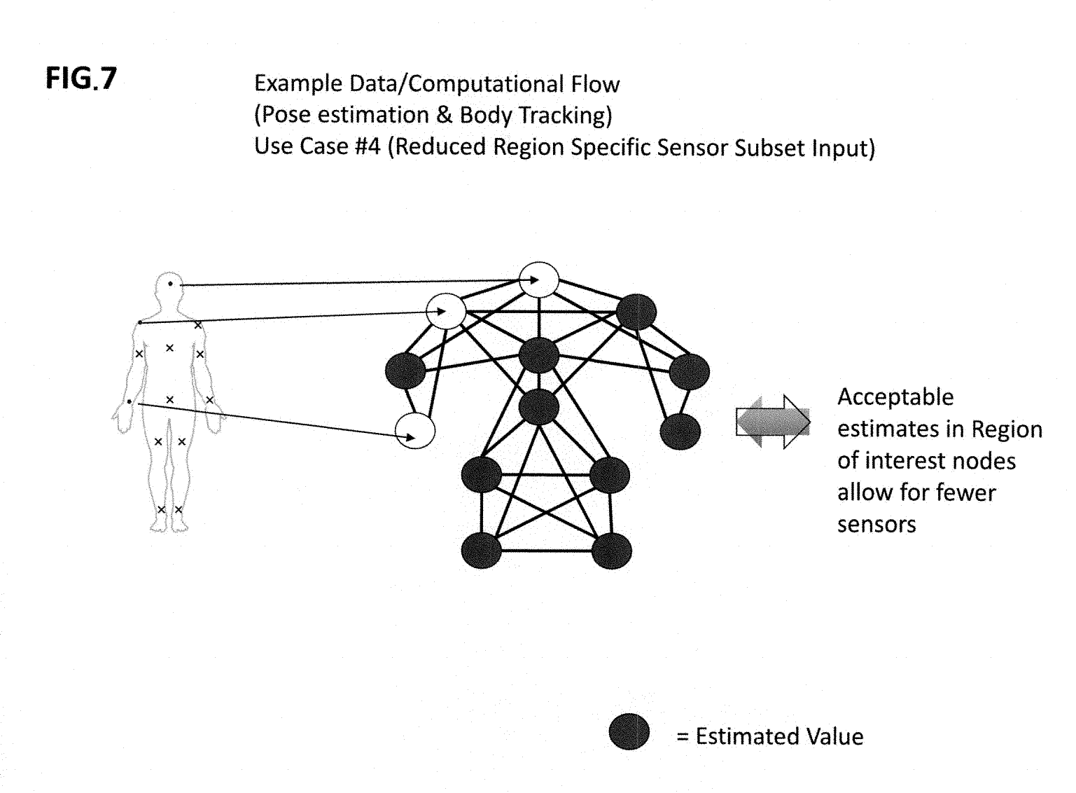

A number of embodiments above describe situations in which there are a reduced number of sensors. A technique to reduce the number of sensors required to get high quality motion tracking enhances the practicality of the system. FIG. 5, 6 and FIG. 7 show region specific sensor subset input with the non-sensored or unmeasured nodes being estimated.

In the context of a network of wearable motion sensors, Predictive algorithms and machine learning techniques such as Probabilistic Graphical models, Neural Networks, Support Vector Machines, nonlinear regression algorithms, or K-Nearest Neighbors can be used to determine the mathematic relationship between the motions and positions of all of the nodes in the network. The system creates a model of the output from each sensor based on the output of all of the other sensors in the network. If one or more sensors are removed from the network, the system uses the learned network relationships to estimate the state of the missing sensor(s). The removal of the sensor may be intentional, in which case the user is able to wear fewer sensors for a given level of motion tracking precision. The removal of the sensor may also be unintentional due to sensor failure, communications dropout or the like. In this case, the network sensor state estimation provides fault tolerance.

An example embodiment of the sensor removal system would comprise a motion capture system, a computer with graphical interface (inclusive of supporting components for data transfer between motion capture system and computer), and a machine learning system. The Machine learning system would consist of a data preprocessor, a learning algorithm, and a data post-processing system.

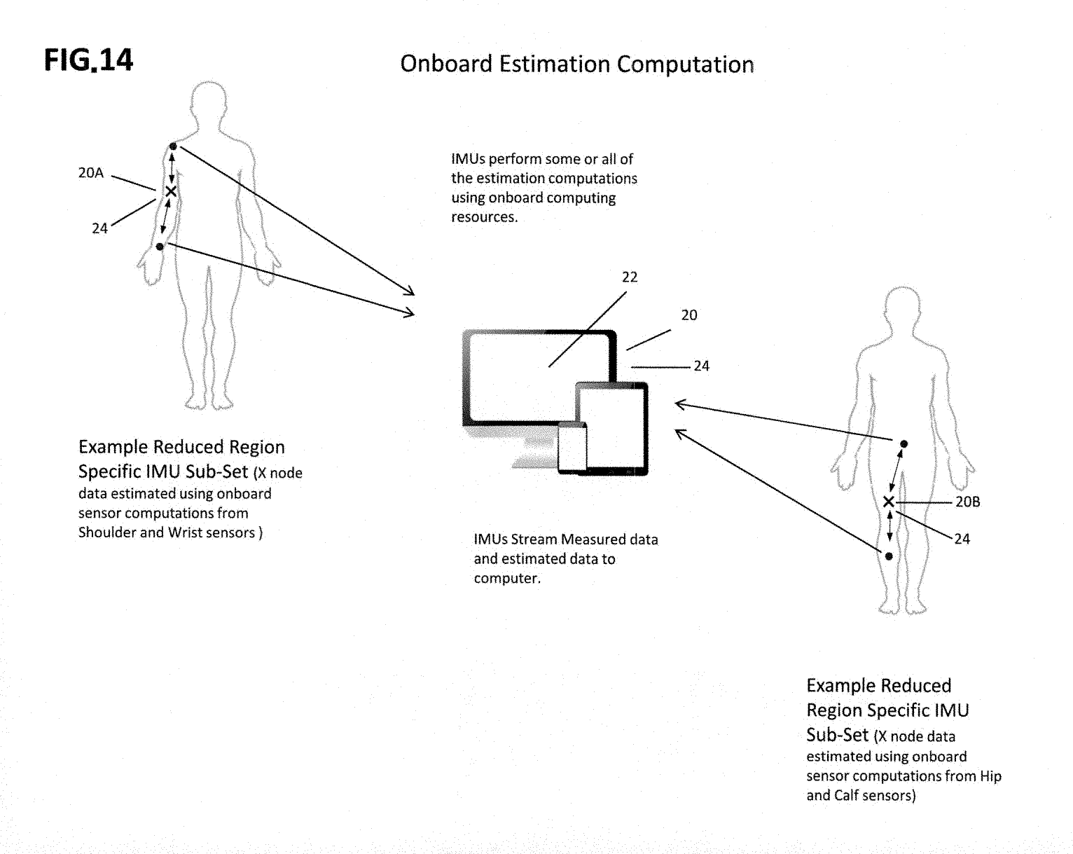

Referring to FIG. 14, the motion capture system reports human body position data through the data transfer system to a main computer 20 (which may be a fixed computer, tablet, personal device or any other suitable device as defined herein) with a graphical interface, and to the machine learning system. The computer may have a recordable medium 24 having instructions for the program with executable code as defined herein. The computer 20 with graphical interface displays an avatar with movements corresponding to the motion capture system. In parallel, the motion capture data is routed to the machine learning system during the learning phase. The machine learning system pre-processes the data and then passes it to a learning algorithm for analysis. The learning algorithm uses the motion capture data to create a predictive system which may also be practiced with the main computer 20 or another computer. The predictive system is recorded on the medium as described herein with instructions as set forth herein. This predictive system is able to take a reduced set of motion capture data and then estimate the orientation(s) of the missing motion capture nodes.

Referring to FIG. 14, a first processor 20A and even a second processor 20B may be coupled to and supported by the tracked person (or moving system). The first processor 20A is carried and supported by the moving system which is in communication (hardwire or wireless) with a first sensor and up to all of the sensors. The first processor 20A is shown in communication with a shoulder sensor and a lower leg sensor which produce motion data that, together with the relationship, are used to estimate the motion data for the upper arm. Similarly, the second processor 20B may be used to estimate the upper arm motion using a torso (which includes the hip as defined herein) sensor and a lower leg sensor (both being in wireless or hardwire communication with the second processor 20B). The first and second processors 20A, 20B transmit the motion data and the estimated data to the main processor 10 which produces data for display of the tracked person (or avatar) as described herein.

Furthermore, consider a motion capture system consisting of IMUs on a subjects' chest, upper arm, lower arm and hand (4 IMUs). With all 4 IMUs reporting data via the data transfer system to the computer with graphical interface and the Machine Learning system, the graphical interface displays an avatar with motion corresponding to the reported data from these four IMUs. The data from these 4 IMUs is also passed to the machine learning system for processing and analysis and for the creation of a predictive system. If, subsequently, only three IMUs are present on the chest, lower arm and hand (3 IMUs with the upper arm IMU not present), the predictive system can be used to estimate the orientation of the sensor not present (in this case the upper arm). This method extends to an arbitrarily large number of IMUs in a motion capture system. The resultant predictive system can then be use to estimate the orientation of an arbitrary number of missing IMUs.

FIG. 12 and FIG. 13 show a specific embodiment of the Machine learning system would consist of a data pre-processor, a K-Nearest Neighbors (KNN) algorithm and a data post-processor. The data transfer system passes quaternion orientation data from each IMU to the data pre-processor, which performs steps to analyze and prepare the data for the KNN Algorithm. The KNN algorithm is used to create a predictive system for later use in estimating the orientation of missing IMUs. The data post processor analyzes the estimated motion data generated by the KNN algorithm and prepares them for use by the computer with graphical interface for display using an avatar, as well as for later mathematic analysis.

The KNN has several advantages for use in this context. First, there are natural, well defined, limits on the Feature Space due to the nature of human body kinematics and the limited range of motion of the human body. Further, each human body has unique patterns of motion and tends to re-visit sets of orientations. This makes KNN particularly useful at essentially learning a “library” of motions associated with a specific individual. KNN can attain an arbitrary level of accuracy given a robust example set.

Experimentation has also shown that once recorded, an example feature set can be reduced in size using a random sampling technique such as simple random sampling, Monte Carlo methods, stratified sampling or cluster sampling. Given a sufficiently representative sub-sample of the original example set, there is very little change in quality of the predictive capability of the KNN algorithm. The sampling rate is an important consideration in this technique. The higher the IMU sampling rate, the more the data set can be reduced. This technique can also be applied to other Machine Learning algorithms such as Probabilistic Graphical Models (PGM), Neural Networks, Support Vector Machines, and non-linear regression. These techniques will also work well with a carefully reduced example set. The benefit of sample set reduction is a reduction in required processing time and a reduction in storage requirements.

A baseline KNN algorithm configuration has proven to be effective at predicting the orientation of missing IMUs given the remaining IMU data. Minimal Pre-processing is required because each IMU reports its orientation as a unit quaternion. We are guaranteed that each unit quaternion coefficient (w, x, y and z for the quaternion w+xi+yj+zk) is between negative-one and positive-one. This reduces the need for normalizing the data prior to KNN. Euclidean Distance can be used as the distance measure for assessing similarity of two samples. The simple Euclidean distance between vectors in the feature space is simple to implement, gives good results and is computationally efficient. A minimum of 2 neighbors (k>=2) must be used to assure an accurate prediction. The use of more neighbors acts to reduce noise and smooth the prediction output. A voting system is used that assigns a weight to each neighbor proportionate to the inverse of the distance from the test point. This weight is then used to interpolate a predicted value if used in a regression, or vote on the appropriate class output if used in a classifier. Because human motion is continuous, interpolation or neighbor class voting gives good results.

Minimal post processing of the estimate is required if the KNN algorithm is used as a classifier. In this case, output of the algorithm is a unit quaternion (determined by voting as described above), and can be used immediately by the rest of the computer system with graphical interface. If the KNN algorithm is used as a regression predictor (with continuous output) then it is possible for the predictor to generate an estimated quaternion which is not unitary (the norm is not equal to one). In this case, the output estimate must be converted to a unit quaternion.

Enhanced Performance KNN

Even though a baseline KNN system has proven to be effective at predicting IMU orientation in human kinematics, we have found that there are a number of ways to improve the accuracy and effectiveness of the system. Combinations of the following techniques have led to improved accuracy, and improved model stability and robustness.

Preprocessing

Normalization and Scaling of the IMU data leads to better predictor performance (see FIG. 13). The quaternion coefficients (w, x, y and z for the quaternion w+xi+yj+zk) can be normalized prior to use in the KNN.

Additionally, the quaternion coefficients can be scaled to the range −1 to 1 prior to passage to the KNN algorithm. Even though the quaternion coefficients are all between −1 and 1 already, some coefficients have generally smaller values and ranges than others. This leads to a disproportionate effect on the distance measurement. Coefficients with small values are under-represented in the similarity measurement, while coefficients with large measures dominate the calculation leading to over-emphasizing certain coefficients. The IMU data set may also be reduced in size using a carefully constructed random sampling technique. If the original data set is a robust representation of the feature space with a high sample density, then the data set can be culled using a random sampling technique with little change in the resultant KNN algorithm predictive performance.

Reduction of Dimension

Since the IMUs pass the data as four Real numbers for each reported quaternion. The baseline KNN system treats each coefficient as a separate feature when in fact the quaternion can and should be viewed as a cohesive entity describing a particular rotation orientation. By treating each quaternion as a cohesive unit, you realize a 4 to 1 dimensionality reduction. With a large number of sensors this becomes important to avoid the “curse of high dimensionality” where the distance between high-dimensional vectors becomes difficult to differentiate. As a case in point, with 11 body IMUs worn simultaneously, the feature space would have 44 dimensions without dimensionality reduction and a much more tractable 11 dimensions with reduction. This dimensionality reduction requires the use of an appropriate distance measure that are mathematically compatible with the quaternion construct.

Alternate Distance Measures

While Euclidean distance has proven effective as a distance measure in our implementations of the KNN algorithm, alternative distance measures have proven even more effective.

Minkowski Distance

By varying the exponent parameter p of the Minkowski distance measure given by d=^{P}\sqrt{(xᵢ−xⱼ)^{P}+(yᵢ−yⱼ)^{P}+ . . .}, one can manually tune the algorithm to more accurately gauge distance and similarity.

An even more effective technique is to use a learning algorithm such as gradient decent to find the optimal value of p in Minkowski distance algorithm.

Cosine Distance

If the quaternion coefficients are viewed as a cohesive entity (that is a quaternion's 4 coefficients w, x, y, and z are kept together to define one feature), then the Cosine distance becomes an extremely effective and appropriate distance measure. The Cosine distance in this context is the angular difference between two quaternions in 4-dimensional hyperspace (3d axis of rotation, and the magnitude of rotation). As pointed out above, combining the 4 coefficients effectively results in a 4:1 dimensionality reduction. Using the cosine distance between quaternion lets us operate in the lower dimensional space, and compare quaternion in a mathematically appropriate way.

Large Margin Nearest Neighbor

Another alternative is to generate an optimal distance measure using the Large Margin Nearest Neighbor algorithm. Gradient descent or similar is used to find the optimum distance measure for the given feature space. The algorithm finds an optimal Pseudo-metric, which is then used by KNN to evaluate similarity of points. The optimal pseudo-metric will outperform other distance measures but is more computationally costly to find and possibly to use.

Alternate Neighbor Voting/Blending Techniques

The weighting and voting system described above can be enhanced by more optimized methods to produce a better estimate. For example, the Geometric or Harmonic Mean, can be used instead of the simple arithmetic mean.

Spherical Linear Interpolation

In the baseline regression case, the K Nearest Neighbors can be combined using the weighting methodology previously described. Neighbors closer to the test point are weighted more heavily than those further away. The weighting methodology interpolates between the estimates, favoring the neighbor with the nearest distance. Simple Linear interpolation can be used with good results but it is far more appropriate to use Spherical Linear interpolation. Unit quaternions can be thought of as spanning the surface of a hypersphere of radius one. If simple linear interpolation is used, the resultant interpolations will be “below” the surface of the hypersphere. Specifically, resultant interpolated quaternions will have not have norm=1. Using spherical interpolation insures the resultant estimates are on the Unit radius 4-dimensional hypersphere. Further, for K>2, SLERP can be used to find the centroid of the area defined by the nearest neighbors on the hypersphere.

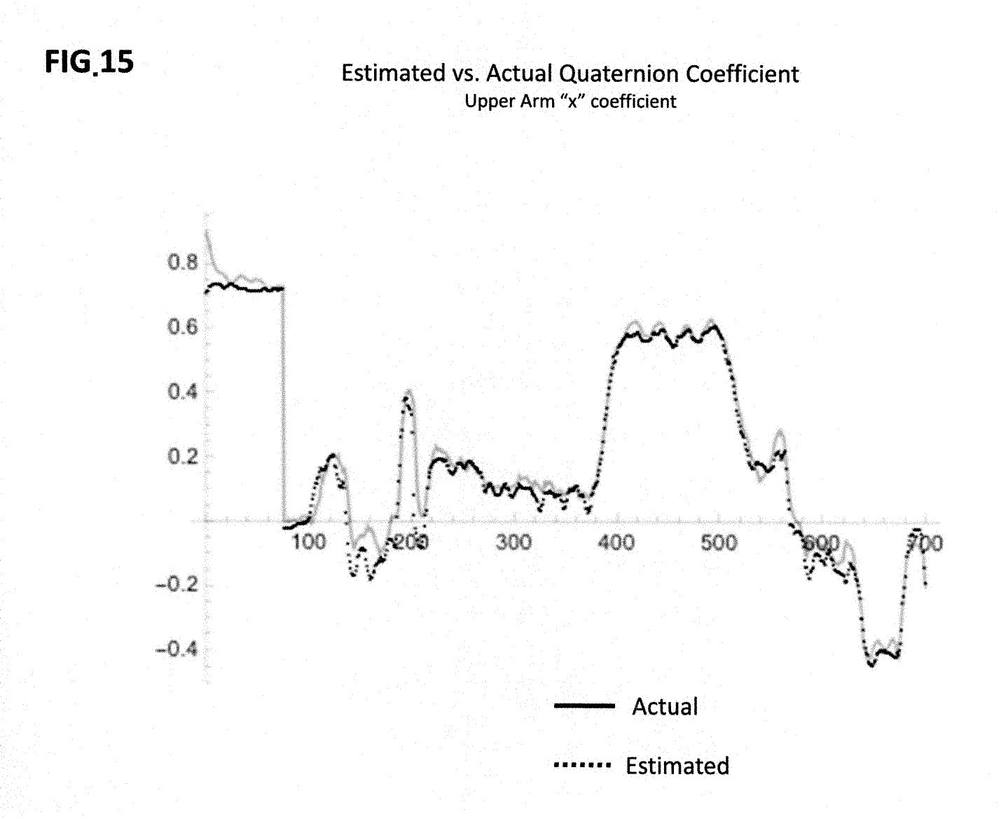

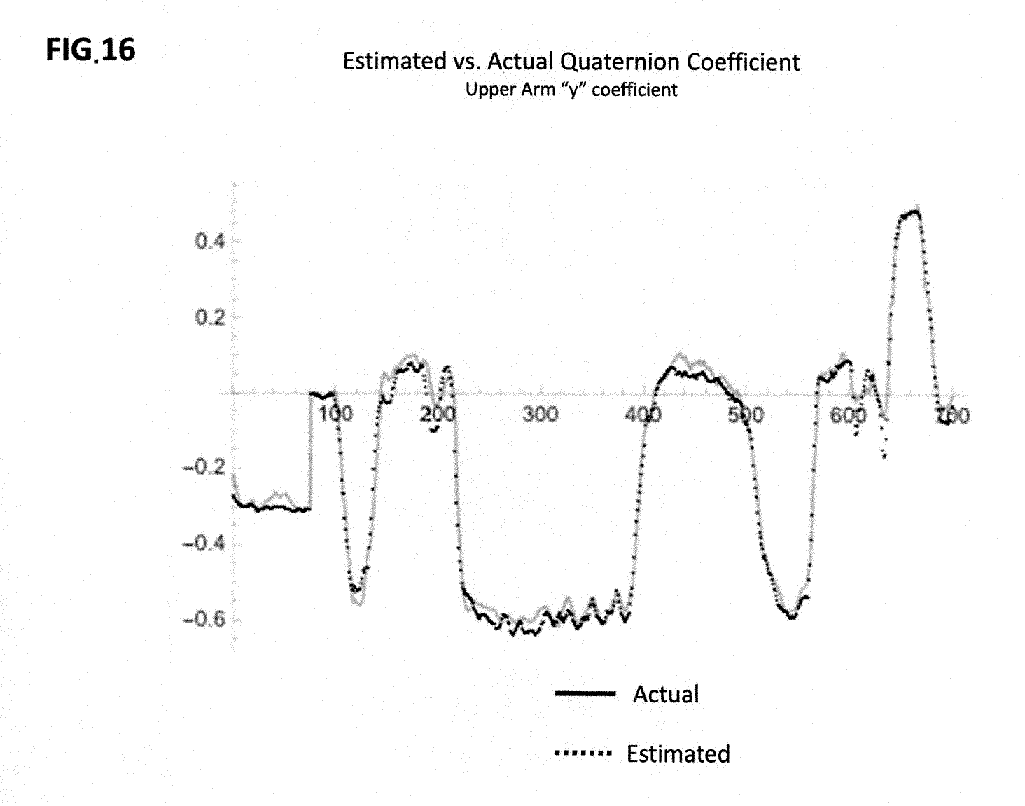

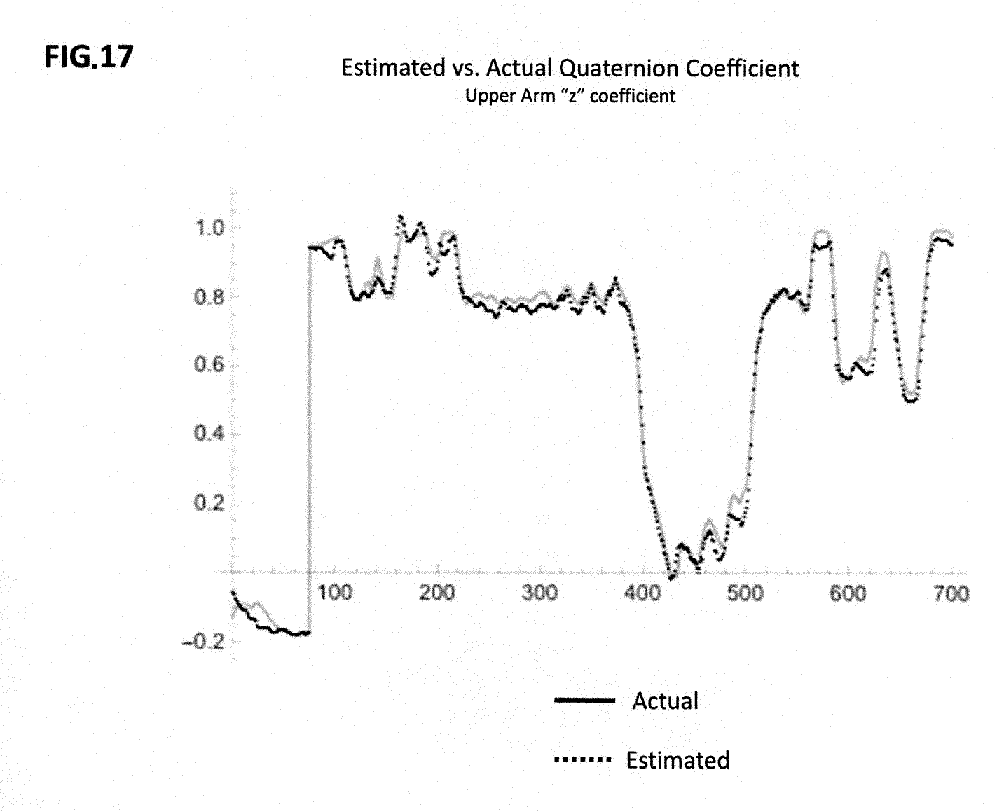

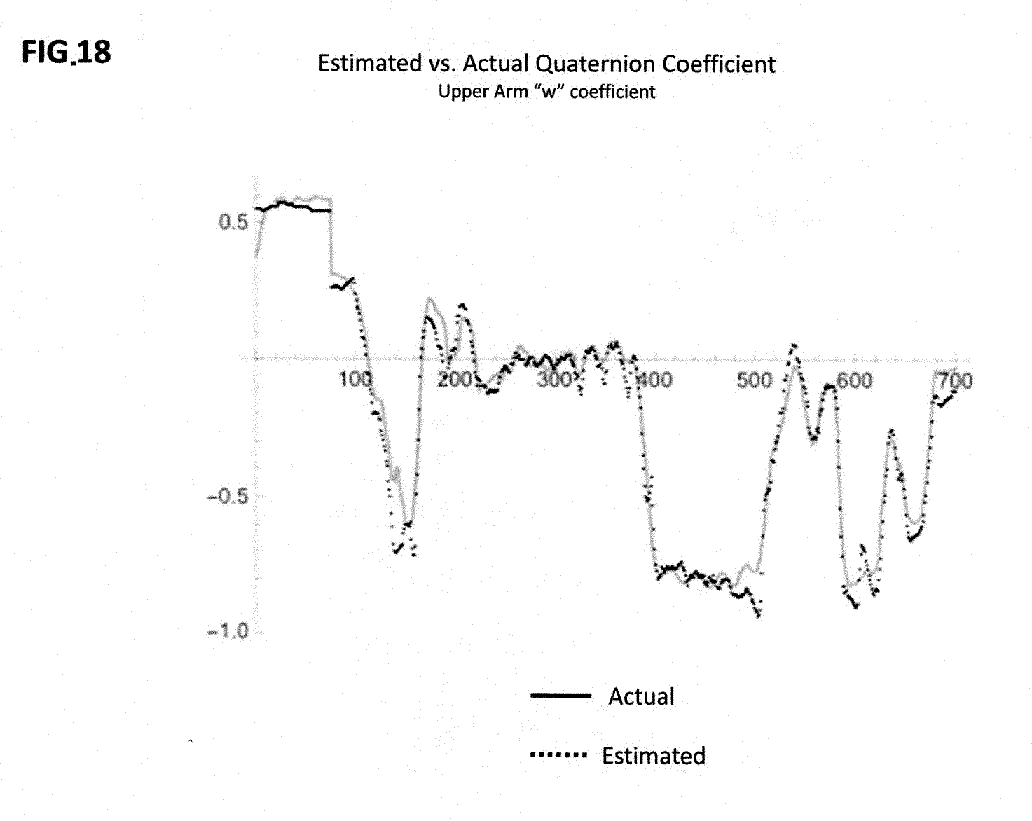

As mentioned above, other algorithms may be used such as a neural network to form the relationship. Referring to FIGS. 15-18, a comparison of each coefficient for an orientation in quaternion form to the coefficient derived from measurements at the estimated location is shown. The neural net provides a good estimate of the motion as can be seen by the correlation to the coefficients derived from measured values.

Advanced Post Processing

Unity Check

If the KNN algorithm is used for regression, then as previously stated, the output may not be a unit quaternion (that is the coefficients generated by the predictor may not have a norm equal to one). The deviation from a norm of one can be used as a feedback metric to optimize the particular parameters for the KNN configuration (selection of K, Distance measure used, etc.). The consistency of the output quaternions norm becomes a figure of merit for the quality of the KNN system.

Extent Check

Since the human body has a finite range of motion, there are limits on the acceptable and reasonable estimates for the predictor to produce. After the KNN algorithm computes a predicted value, the system compares the predicted value with known maximum orientations. If the prediction exceeds these values appropriate actions can be taken.

Absolute Angle Joint Measurements

As a result of the sensor orientation being reported in quaternion form format, Algebraic operations can be performed on data from adjacent sensors (thigh and calf, upper arm and lower arm or chest and upper arm for example), that compute the angular difference between these two adjacent sensors. The angular difference between two adjacent sensors can be reported in a number of ways. Quaternion algebra can be used to solve for the angular difference expressed in Euler angles, which decompose the angular difference into a sequence of 3 component rotations in the x, y, and z axes as referenced to a common frame of reference. The angular difference between two adjacent sensors can also be expressed as a single angle of rotation about a single axis. Euler's rotation theorem states that any rotation or sequence of rotations of a rigid body or coordinate system about a fixed point is equivalent to a single rotation by a given angle θ about a fixed axis (called Euler axis) that runs through the fixed point. Thus, a sequence of 3 rotations about the x, y, and z axis is mathematically equivalent to a single rotation about some single axis. Quaternion algebra allows us to efficiently compute this single rotation axis and the angle of rotation about this axis. In the context of human body kinematics this is important because the axis of rotations for human joints rarely if ever remain static. That is, as a limb is moved, the axis of rotation does not remain fixed. For example, when the leg is extended, the knee joint does not rotate like a hinge confined to one axis of rotation. Instead, the axis of rotation varies as the leg is extended. This introduces a significant problem for physicians and therapists trying to measure the angle of rotation of a joint using traditional Goniometry techniques. They are forced to manually pick an axis of rotation, with no guarantee of picking the same axis from measurement to measurement. These inconsistent measurements give a less accurate representation of the subject's range of motion for example. Conversely, using a single angle of rotation about a single axis computed dynamically always represents the absolute magnitude of the angular difference between two sensors. This form of measurement is absolutely consistent, and allows a physician or therapist to get a reliable and accurate baseline measurements, follow on measurements, range of motion, range of motion improvement or degradation, all intra and inter subject comparisons, and any other form of comparative measurement.

Virtual Reality

Virtual Reality (VR) display technology is reaching a high level of maturity. Modern VR displays immerse the user in an immersive experience with a high degree of realism. One of the main ways in which VR displays are not realistic is their lack of representation of the users own body in the simulation. For example, if a user wearing a VR headset turns their head to a position which would allow them to see their own arm, they currently cannot see their arm position represented accurately in the field of view. This lack of ability to see your own body quickly destroys the illusion of realty in the system. Furthermore, the lack of accurate 1st person body display in the simulation also limits the ability to interact with other users in the virtual reality environment.

The method and system for measuring and tracking a user's movement using a set of sensors described above can be integrated into a VR system to enhance the Virtual Reality experience and enable multi-person interaction in the Virtual Realty environment. The network of body measurement sensors reports the angular relationships in quaternion form format. These quaternion data are currently used to drive an animated avatar displayed in 2 dimensions on a screen. The same data can be used to drive a 3d avatar in a VR environment.

The network of body sensors can be initialized to report angular position relative to a fixed world frame of reference using Magnetic north and gravity down as references for example. Likewise, VR headsets and display devices use a fixed frame of reference during a session to orient the user. The body sensor network frame of reference can be aligned with the VR display device frame of reference using algebraic quaternion transformations. In this way, movements of the human body as reported by the body sensor network can be shared with the rotation information in the VR system. Specifically, one can use quaternion algebraic operations to translate the body sensor information for display in the VR environment. The VR user would then be able to accurately see his own body position and body position of others in the VR display. VR environments use cameras and lasers to track the position of at least 1 fiducial on or held by the participant. This fiducial indicates where the participant is but has no way of knowing where the rest of their body is. Currently, systems can use the standard cameras and lasers etc. to approximate the body position within the VR environment. These types of systems are costly and sometimes inaccurate. This invention allows very accurate projection of the participant into the VR environment without the cost and inaccuracy by numerically joining the body tracking modalities to provide a much more realistic VR experience at a fraction of the cost of a true camera body tracking system.

Additionally, a system described above would provide a benefit to current virtual reality environments that require line of sight from fixed cameras or lights to the sensors or emitters worn by the user. If the path is blocked between the receiver and emitter, the position and accuracy of the user is no longer known. Having individual sensors on a body that are not dependent on emitting or receiving line of sight can be advantageous because they may be less susceptible to such problems. Therefore, in situations where there is a potential for such ‘shadowing’ the system described herein provides certain advantages. For example, if there are multiple users then one user cannot block the other user's connection to the base system.

Furthermore, sensors that include accelerometers or gyrometers may be used to control features within a virtual environment. For example, the user may shake their elbow to the side in order to bring up a certain menu or any other command. In virtual environments that use handheld controllers, the user typically uses buttons or other controls to perform certain tasks or navigations. In the system described herein, the users body positions or movements may be used for similar commands and therefore the user may not need a handheld controller.

The system may additionally identify certain ‘standard’ body positions based on the user's orientation. For example, in certain first person shooting games there may be a discrete number of body positions the virtual character can take. Rather than trying to monitor an infinite number of body positions, the virtual reality environment may create a discrete number of positions, such as crouch, crawl, walk, run, stand, etc. As the user moves, the system described herein may identify when the user's body is close to one of the discrete positions and identify this position to the virtual environment which in turn moves the virtual character into this position. This may be useful for improving performance of the virtual environment and not needing to relay every piece of sensor information to the virtual environment. Additionally, certain users may not be able to achieve certain positions based on their own physical limitations. Therefore, a system which can infer discrete positions based on the user's body orientation would be useful.

The system may also be used with a variety of feedback mechanisms. For example, one or more sensors may include haptic feedback mechanisms to the user such as vibration. The individual sensor blocks may each have a vibrating motor which can provide more specific feedback to the user based on the virtual environment. In the case of physical therapy, the virtual environment may provide haptic feedback to a sensor block which is located on a user's lower leg to indicate to the user that they need to lift their lower leg more. Additionally, within virtual environments the feedback may be provided to specific locations such as an elbow when the virtual character has come in contact with an object within the virtual environment. This may improve the immersive nature of the virtual environments.

Referring to FIG. 14, the present invention may be carried out with the first processor carried (supported) by the person/user which may perform estimations of the aspect and transmit the motion data and the estimation to the main processor which is independent of the moving system and may be a fixed computer, laptop or a dedicated device. The moving system may include several processors with each coupled (preferably by hard wire) to one or more sensors for estimating the aspect based upon a reduced or regional set of sensors. For example, the first processor may define a relationship between quaternion orientation data of a lower arm, a shoulder and a torso sensor and the upper arm to estimate a quaternion orientation for the upper arm. When an upper arm sensor is provided as well, the first processor may compare the estimated aspect with the aspect derived from measurements of a sensor coupled to the upper arm of the moving system. As can be appreciated, the moving system may even have four processors with one each for the upper arms and legs in accordance with the invention described herein with each estimating motion data for part of the body. An advantage of providing one or more processors on the moving system or each of the moving systems in a populated environment. The distributed processing (estimation, comparison and modification) at each of the moving system unburdens the main processor which provides advantages when multiple moving systems are within the same virtual environment or are being assessed at the same time while also taking advantage of regional sets which can accurately estimate motion data with the regional sensors.

The inventive concept may be embodied as computer-readable codes on a non-transitory computer-readable recording medium. The non-transitory computer-readable recording medium includes any storage device that may store data which may be read by a computer system. The computer-readable codes are configured to perform operations of implementing an object arrangement method according to one or more exemplary embodiments when read from the non-transitory computer-readable recording medium by a processor and executed. The computer-readable codes may be embodied as various programming languages. In addition, functional programs, codes, and code segments for embodying exemplary embodiments described herein may be easily derived by programmers in the technical field to which the inventive concept pertains. Examples of the non-transitory computer-readable recording medium include read-only memory (ROM), random-access memory (RAM), CD-ROMs, magnetic tapes, floppy disks, and optical data storage devices. The non-transitory computer-readable recording medium may be distributed over network-coupled computer systems so that the computer-readable codes are stored and executed in a distributed fashion. As used herein, the terms “having” “has” “includes” “including” “comprises” and “comprising” (and all forms thereof) are all open ended so that A “having” B, for example, means that A may include more than just B. Finally, all techniques, methods, systems and devices described in connection with a particular algorithm all of the techniques, methods systems and devices described are expressly incorporated into all other suitable algorithms described herein. For example, the preprocessing steps are described in connection with using KNN to define the relationship and use of the preprocessing may be incorporated into any other suitable algorithm such as a neural network.

Although embodiments of various methods and devices are described herein in detail with reference to certain versions, it should be appreciated that other versions, embodiments, methods of use, and combinations thereof are also possible. Therefore, the spirit and scope of the invention should not be limited to the description of the embodiments contained herein. Furthermore, although the various embodiments and description may specify certain uses the invention may be applied to other suitable applications as well.

1-69. (canceled)

70. A system for assessing an analyzed motion of a moving system, comprising:
a non-transitory computer readable medium having executable computer instructions thereon, the instructions include the steps comprising defining a first relationship between motion data for an aspect of a moving system corresponding to a first assessed location and motion data corresponding to a first sensor location, estimating the aspect for an analyzed motion of the moving system captured by a first sensor in at least approximately the same corresponding position as the first sensor location, the estimating using the relationship and motion data for the first sensor to estimate an estimated aspect at the first assessed location of the moving system.

71. The system of claim 70, further comprising:
a processor which receives the captured motion data from the first sensor for the analyzed motion, the processor carrying out the instructions on the medium.

72. The system of claim 70, further comprising:
a non-transitory memory which records the analyzed motion data for the first sensor and the estimated aspect.

73. The system of claim 70, wherein:
the instructions on the medium for the defining uses a predictive algorithm to form the relationship.

74. The system of claim 70, wherein:
the instructions on the medium for the defining uses a plurality of motions of the moving system and a predictive algorithm to form the relationship.

75. The system of claim 70, wherein:
the instructions on the medium for the defining includes processing the motion data of the analyzed motion to a reduced motion data size in quaternion form.

76. The system of claim 70, wherein:
the instructions on the medium for the defining uses a neural network which forms the first relationship using a plurality of motions of the moving system.

77. The system of claim 70, wherein:
the instructions on the medium for the defining forms the relationship between quaternion coefficients of the aspect and quaternion coefficients of the first sensor location using motion data corresponding to the first sensor location and to the assessed location for a plurality of motions of the moving system.

78-80. (canceled)

81. The system of claim 70, wherein:
the instructions on the medium for the defining is carried out with the aspect being an orientation in quaternion form and the first sensor is an inertial measurement unit.

82. The system of claim 70, wherein:
the instructions on the medium for the defining step is carried out with the moving system being the same as the moving system.

83. (canceled)

84. The system of claim 70, wherein:
the instructions on the medium include comparing the estimated aspect to a measured value derived from the motion data of the analyzed motion.

85. The system of claim 70, wherein:
the instructions on the medium for the comparing changes in a person in physical rehabilitation during the analyzed motion while the relationship is formed from a plurality of motions captured earlier than the analyzed motion.

86-94. (canceled)

95. The system of claim 70, wherein:
the instructions on the medium for the estimating includes estimating contemporaneous with the analyzed motion.

96-102. (canceled)

103. The system of claim 70, wherein:
the instructions on the medium include checking for an error in the estimated aspect by calculating whether the estimated aspect is a unit quaternion.

104-113. (canceled)

114. The system of claim 70, wherein:
the instructions on the medium include reducing the motion data for the analyzed motion in size using Random sampling techniques before the estimating.

115-126. (canceled)

127. The system of claim 70, wherein:
the instructions on the medium include transmitting the estimated aspect to a main processor from a first processor hardwired to the first sensor and coupled to the moving system, the instructions for estimating being carried out by the first processor.

128. The system of claim 70, further comprising:
a first processor in communication with the medium to receive the executable program, the first processor estimates the aspect in accordance with the instructions of the executable program.

129. The system of claim 128, wherein:
the first processor is carried by the moving system.

130. The system of claim 128, wherein:
the first processor compares the estimated aspect with a measured aspect derived from the motion data of the analyzed motion.

131-135. (canceled)

136. The system of claim 128, wherein:
the instructions on the medium include transmitting motion data from the first processor wirelessly to a main processor.

137-146. (canceled)
